(19) United States
(12) Patent Application Publication
(10) Pub. No.: US 2015/0093367 A1
(43) Pub. Date: Apr. 2, 2015

(54) ACUPOINT INJECTION THERAPY USING AN ENRICHED CELLULAR MIXTURE

(71) Applicant: GLOBAL HEALTH SOLUTIONS RESEARCH TRUST, GLEN COVE, NY (US)

(72) Inventor: BILL AKPINAR, GLENCOVE, NY (US)

(21) Appl. No.: 14/500,359

(22) Filed: Sep. 29, 2014

Related U.S. Application Data
(60) Provisional application No. 61/883,493, filed on Sep. 27, 2013.
(60) Provisional application No. 61/935,118, filed on Feb. 3, 2014.
(60) Provisional application No. 61/944,767, filed on Feb. 26, 2014.

(51) Int. Cl. A61K 35/14; A61K 35/16; C12N 13/00

(52) U.S. Cl. CPC A61K 35/19; C12N 13/00; A61K 35/14; A61K 35/16
USPC 424/93.72; 435/173.1

(57) ABSTRACT

A system and method are provided that use the meridian flow defined in traditional Chinese medicine to target specific regions of the body to treat certain health conditions. A cellular mixture is injected into the body via an acupoint on a particular meridian in order to deliver the mixture to an organ or body region associated with the particular meridian. The cellular mixture can be a stem cell/PRP mixture.

### CROSS-REFERENCE TO RELATED APPLICATIONS

The present application claims benefit of: co-pending Provisional Patent Application No. 61/883,493, filed on Sep. 27, 2013; co-pending Provisional Patent Application No. 61/935,118, filed on Feb. 3, 2014; and co-pending Provisional Patent Application No. 61/944,767, filed on Feb. 26, 2014; those applications being incorporated herein, by reference, in their entireties.

### BACKGROUND OF THE INVENTION

1. Field of the Invention

The present invention relates to a system and method for using a stem cell/platelet-rich-plasma (PRP) mixture to treat certain conditions and, more particularly, to a system and method that treats a condition using a stem cell/PRP mixture that is injected into the meridian system used by traditional Chinese Medicine.

2. Description of the Related Art

The theory of Qi and movement of energy is a foundational cornerstone of Chinese Medicine and has been in use for over thousands of years. Chinese medicine is based on a tradition of more than 5,000 years and includes the use of herbal medicine, acupuncture, exercise and other components. Balances and imbalances in the body are characterized by normal or abnormal movement of “Qi” (or life energy) throughout the body.

While Chinese Medicine accepts and acknowledges the existence of traditional circulatory nervous and lymphatic channels, it also defines a system of “meridians” (i.e., channels). This is an independent, unique system, but a system which works parallel to the normal nervous and circulatory system of the body. Acupuncture points or “acupoints” are points found along the meridian system. It is believed that there are over 365 acupoints in the human body. These acupoints are different from normal tissue histologically, morphologically and neurologically and are approximately one 1 cm in diameter. In Traditional Chinese Medicine these points, when stimulated via acupuncture have been shown to demonstrate an effect by helping induce movement of Qi, or life energy, through the related meridian system. Thus, the acupoints are seen as “entrance ramps” into a highway of rivers or channels flowing within the body, which are linked to, but independent from the circulatory and nervous system. In effect, a meridian is a path through which life energy, known as Qi, flows. The 365 known acupoints are located along the 20 major meridian systems including the 12 regular meridians (or 12 regular channels), with each meridian corresponding to a particular organ, bringing life force (a fluid flow in this invisible “underground flow pathways of the body” that are unique and comprise their own independent circulatory system).

Up until recently, the meridian pathways were believed to be “metaphoric” or a spiritual representation of Chinese Medicine, which is an ancient healing science. However, in 1985, Pierre De Vernejoul, a researcher at the University of Paris, used radioactive isotopes to authenticate the existence of meridians. De Vernejoul injected the radioactive isotopes (mainly technetium 99m) into classic acupuncture points. Then, using gamma camera imaging, De Vemejoul tracked the movement of the isotope through the meridian system to what was originally described as the target organ (i.e. the kidney in the kidney meridian, the bladder in the bladder meridian, etc.). The amazing results of the study verified that there is indeed a separate channel flow through these meridians that, when stimulated, would be capable of bringing various substances to the target organ. The injected isotopes were demonstrated to travel a distance of about 30 cm within 4-6 minutes.

In order to test De Vemejoul's findings, radioactive isotopes were injected outside of any acupoint, and the results were dramatic. The isotopes injected outside the acupoints diffused in a circular manner and just dissipated into the adjacent tissues, rather than following the pathways described by the ancient Chinese as leading to a target organ. Therefore, when the isotope was injected into a non-acupuncture point, the effect found by De Vernejoul was not demonstrated. In other words, the isotopes did not travel to a “target organ” or end organ of the meridian system, in contrast to what was observed when the isotopes were injected into an acupoint. The validity of the pathways miraculously described in detail by the eastern monks thousands of years ago, was now verified by science. De Vernejoul's research verified and validated the theory that the meridians are a continuum of liquid crystalline water bond collagen fibers running through the entire body and having what has been termed “dielectric and conductive properties”. Thus, it has been validated that there are separate, connective tissue pathways within the different tissues of the body in which interstitial fluid flows.

Therefore, these meridian channels provide an efficient system of migratory tracts, mainly due to durotaxis, for not only interstitial type fluid, but also for mast cells, fibroblasts and other cells that migrate and carry out a number of physiologic functions. Interstitial fluid (or tissue fluid) is a solution that bathes and surrounds the cells of multi-cellular animals. These are cells which have been shown to exist within the interstitial pathways known as the meridians. They can be described as “underground rivers” of energy flow. It is the main component of the extra-cellular fluid which also includes plasma and trans-cellular fluid.

Interstitial fluid bathes the cells and tissues. This interstitial fluid has been found to contain not only sugars, salts, fatty acids, amino acids and cellular components, but, amazingly enough, also formed cells, including platelets and white blood cells. Consequently, the interstitial fluid provides a viable means of delivering material to the cells intra-cellularly. Pierre De Vernejoul's findings were further validated in the Italian-Chinese School of Acupuncture by Dr. Enrico Lonordo and Professor Achileabbati in 1988. When radioactive technetium (tc-99-m) was injected into these acupoints in the verification research done at the Italian-Chinese school, and migration of the radioactive tracer was followed by camera studies, the findings paralleled what Pierre De Vemejoul had initially discovered.

The meridian flow is based on a “meridian clock”, i.e., a 24 hour cycle related to flow pattern of Qi or this interstitial fluid described within the meridian system. Each of the 12 meridians has a dual flow, kind of a one-way street being reversed at a certain time of the day, it comes and goes marking the 24-hour-a-day cycle. The flow from each of the meridian systems (i.e. the liver, the stomach, the kidney, etc.) is based on two hour flow cycles during which the flow is maximized in the direction of the meridian. The basis of the flow is that these 2 hour cycles are programmed by nature in such a way as to complement the natural course of events in the body, i.e., ingestion of food during the working hours, breakdown of food and house cleaning in the sleep hours, elimination in the morning hours immediately after waking. For example, from 7-11 AM the spleen and stomach organs which relate to the “earth meridian” are active, the time to rise from sleep and breaking the overnight fast. At 7:00 AM the large intestine is active in motivating the morning elimination, the best time to wake up, therefore, is between 7:00 and 8:00 AM, according to this. The afternoon “fire meridians” of the heart and small intestines open between 11:00 and 15:00 hours; this is the best time to accomplish certain things such as tasks and physical labor. Like the “fire element” described, the “fire” is the energy that fuels the life force during this period. The “water element” meridian will be most active between 3:00 and 5:00 PM and, correspondingly, this should represent the time of relaxation and rest. In light of these and similar observations, the ancient Chinese have mapped out a specific pattern of flow in specific regions which when adhered to, would maximize health. Modern science with its technologically advanced tools has thus verified and validated what the ancient scientists knew.

What is needed is a system and method that utilizes the specific patterns of flow in specific regions to treat certain health conditions. What is further needed is a system and method that utilizes the meridians to provide injected cells to specific regions of the body.

Additionally, there currently exists a need in stem cell/platelet-rich-plasma (PRP) technologies to direct the cellular components to specific “target areas” in the human body. Although administration of the stem cells/PRP mixture can be done directly into the muscles or into the main circulation via intravenous introduction, there is still a challenge to get the mixture to the specific desired areas. Although such administration does elicit a response, which is known as a “signaling response” (after the expiration of the lifespan of the original cells, “signals” are produced to induce other cells to produce in a similar manner), this response is not specifically targeted to a desired area of the body. In the current medical world, various organ systems can only be accessed via surgical access or needles. However, this presents various risks (i.e. opening the brain in order to implant cells which can repair is highly risky and dangerous, or going into the heart or other vital organs can be potentially hazardous and create complications).

What is further needed is a way to direct the cellular components of a stem cell/PRP mixture to specific target areas of the body.

### SUMMARY OF THE INVENTION

The present invention provides a system and method that addresses the needs of the prior art. In particular, the present invention uses the meridian flow defined in traditional Chinese medicine to target specific regions of the body to treat certain health conditions. A cellular mixture is injected into the body via an acupoint on a particular meridian in order to deliver the mixture to an organ or body region associated with the particular meridian. In one particular embodiment of the invention, the cellular mixture is a stem cell/PRP mixture.

Other features, which are considered as characteristic for the invention, are set forth in the drawings and the appended claims.

Although the invention is illustrated and described herein as embodied as acupoint injection therapy using an enriched cellular mixture, it is nevertheless not intended to be limited to the details shown, since various modifications and structural changes may be made therein without departing from the spirit of the invention and within the scope and range of equivalents of the claims.

### BRIEF DESCRIPTION OF THE DRAWINGS

For a fuller understanding of the nature of the present invention reference should be made to the following detailed description taken in connection with the accompanying drawings in which:

FIG. 1 is a simplified flowchart of a method of treating a body part or region of a host, in accordance with one particular embodiment of the present invention;

FIG. 2 is a partial view of a person showing a portion of the meridian system for treating issues related to the head; and

FIG. 3 is a partial view of a person showing a portion of the meridian system for treating issues related to the heart.

### DETAILED DESCRIPTION OF THE PREFERRED EMBODIMENT

The present invention provides a system and method that is based on the movement of Qi through the meridian system, as defined by traditional Chinese medicine. The method involves the delivery of autologous cells (host derived stem cells, taken from the blood of the host) into acupuncture points or “acupoints” along a meridian system. Based on the theories of energy and Qi flow along these meridians, the injected autologous cells are delivered to a “target area” identified using the foundations of traditional Chinese medicine (TCM). Traditional Chinese Medicine views the body, not primarily with reference to anatomical structures, but focuses primarily on the body's functions and movement of energy.

Using the principles of energy movement and body function defined in TCM, the present invention provides a transport pathway, not only for chemical substances i.e. potentially different forms of “chemotherapy”, drugs, herbal preparations, forms of oxygen/ozone, derived in ozone, (including the herbozone technique), but also for the delivery of actual stem cells and platelet-rich-plasma (PRP), by administering a cellular mixture via acupoints to induce the cells to reach the desired target organ. More particularly, by injecting a stem cell/PRP mixture directly into the appropriate acupuncture points, they can be induced via the body's own “Chi flow” system (i.e., the body's “underground rivers”) to travel to the specific target area via the meridian. Therefore, the administered agent (which can be a stem cell, a chemotherapy agent, ozone or any other component) can be induced to travel to the desired region eliciting a physiologic response.

For example, according to one embodiment of the present invention, stem cells from the recipient or host are placed into the heart meridian particularly at the specific two-hour phase when the energy flow is most active in the heart meridian, thus inducing the cells to go to the heart and promoting the repair and production of healthy heart tissue. As can be seen, the precise timing for the optimal time for accessing the meridian is a technical issue to be considered. Using a viable, autologous stem cell extraction method, the cells are taken from the host donor, (therefore minimizing any chance of complications, infection, or rejection, etc.).

Stem cell technologies have been in use for many decades in their various forms. The National Institute of Health and Epidemiologic Studies indicate that in approximately five years, 1 of 3 individuals will develop a form of cancer or chronic disease such as Degenerative Arthritis, Alzheimer's disease, Cardiac disease, Cerebrovascular disease, crippling pain syndromes and diabetes, to name a few. This is the reason so much research has been directed towards the adjunctive use of stem cells and other primordial components of blood as treatment as well as coadjutant for the treatment of these and many other conditions that will impact the future of humanity. Stem cells have the remarkable potential to develop into 220 different cell types in the body during early life in growth. Moreover, in many tissues they serve as a type of internal repair system dividing essentially without limit to replenish other cells as long as the person or animal is still alive. When a stem cell divides, each new cell has the potential even to remain as a stem cell or to become another type of cell with a more specialized function, such as muscle cell, blood cell or a brain cell.

One particular embodiment of the present invention uses adult or somatic stem cells, a relatively rare, undifferentiated cell found in many organs and tissues with limited capacity for both self-renewal and differentiation. In a human being, adult stem cells are available to divide when needed and can give rise to mature cells. They have characteristics, shapes, sizes, specialized structures and functions of a particular tissue. A way to visualize the potential of an adult stem cell is to picture a damaged heart; when the muscle is weakened, this can happen after a lifetime of poor habits such as poor lifestyle, compromised diet, smoking, or it can simply be a birth defect. In one particular example, adult stem cells, which are extracted from blood and processed, could, in theory, be injected into a damaged heart muscle. After this injection, the cells will “sense” where there is a need for a specific heart muscle cell, and will generate these needed cells, thus resulting in a stronger, healthier heart muscle.

In a process known as “signaling” (where these cells induce other cells to behave in a certain way), cells which have been made to travel to a desired region of the body will “signal” other cells to be healthier, long after the original cells have died off. This can also be applied to any of the body's various organ systems. Pain and diminished functional integrity are two of the main factors that will induce a person to seek medical care. Consequently, a major focus of the present invention is related to pain management. Using a combined compound of stem cells and platelet-rich-plasma (PRP), the method of the invention is a more effective alternative or adjunctive therapy to pain management with virtually no undesirable side effects.

More particularly, the present method utilizes the body's natural healing powers, which results in effective outcome with little hazard or inconvenience to the patient. Platelets resemble sponges when at rest and form branches when activated. Although, it was originally thought that platelets only cause blood clotting, recent discoveries have shown us that activating platelets causes a release of growth factors, a protein essential for the body to heal on its own. These growth factors have a number of functions in a body, and their primary responsibility is to aid in wound and tissue healing. Therefore, when the baseline concentration of PRP and stem cell compound is increased the amount of growth factors also increases significantly enough to result in accelerated tissue growth and recovery. Several of the aforementioned healing factors (found in elevated levels in the stem cell/PRP mixture) that can be expected to show a moderate-high degree of increase will include:
Platelet Derived Growth Factor (PDGf);
Transforming Growth Factor (TGF);
Vascular Endothelial Growth Factor (VEGF);
Insulin-like Growth Factor (ILGF); and
Epidermal Growth Factor (EGF).

One particular embodiment of the method of the present invention utilizes the placement of a stem cell/PRP mixture into, not only the general blood using intravenous introduction, but also into the Chinese meridian system based on need and in conjunction with the principles of traditional Chinese medicine. As discussed above, a meridian in traditional Chinese medicine is also called a channel or path through which life energy, such as Chi, flows. Along these channels there are at least 365 documented acupuncture points or acupoints. If the meridians are viewed as energy highways, the acupoints can be seen as entrance points or on-ramps by which to access these highways through the human body. Chi, or life energy, flows through the meridian, or energy highway, accessing all parts of the body. The meridians have been mapped throughout the body and their flow has been mapped, demonstrated, replicated, and duplicated by use of radioactive materials. As also discussed above, researchers in the 1950s used radioactive technicium, injected directly into the acupoints and demonstrated via high-tech tracking and photography systems that when the radioactive dye is injected precisely into the acupoint, it travels along the meridian system along which the acupoint lies and ultimately reach the “target organ.” The target organ is one of the main areas upon which the effect is exerted. Each meridian has many acupoints along its path. The meridians do not float on the surface, but can be seen as underground rivers flowing throughout the body. As previously indicated, the presence of a vast multitude of meridians and points make virtually any region of the body accessible to treatment without surgical risk. They are primarily located within the interfascial planes (these are the spaces between the fascia and the muscles). The meridians are responsible for the distribution of basic substances throughout the body, just like circulatory system. However, conventional anatomy and physiology has been unable to identify these pathways, which is why radioactive dye tracing was used to document and validate what the ancient physicians knew. The substance that flows through the meridian system is a type of fluid that is believed to be a serous type of fluid and is similar in composition to lymphatic fluid or cerebrospinal fluid. It also may contain cells, as well.

There will now be described one particular method for treating a body part or body region of a host, in accordance with one embodiment of the present invention. First, In accordance with the present method, host derived stem cells are taken from the blood or fat tissue of a host. Any extraction method that has a scientific foundation is acceptable to this end. However, in one particular embodiment of the invention, an Arteriocyte Magellan machine (or an equivalent thereof) is used to extract a mixture of PRP and stem cells. This mixture is ideal because PRP has been demonstrated to contain numerous regenerative and healing properties. Plasma is the liquid part of the blood. Platelets, while at rest, do not show much activity, when activated demonstrate a multitude of regenerative and restorative activity in the cells, tissues and organs of the body.

In one particular embodiment of the method, no more than 90 cc of blood is drawn from the body (in each of three draws). After each draw, the blood is treated with an anticoagulant (selected by the physician) to prevent clogging within the syringe. If desired, citrate may be used as the anticoagulant. However, this is not meant to be limiting, as other anticoagulants can be used, as desired.

Each blood draw will be used to produce a stem cell/PRP mixture after being spun in an arteriocyte Magellan machine or equivalent. The amount of stem cells and/or precise machine used may vary, as desired. Alternately, or in addition, stem cells may be extracted from the patient's mesenchyme (the fat suction using a liposuction technique centrifuged, liquefied, and injected into the stem cell meridian in a similar manner).

Additionally, in accordance with one particular embodiment of the invention, the stem cell/PRP mixture can be enhanced after each draw through the performance of additional procedures. For example, in one particularly preferred method, the stem cell/PRP mixture of each blood draw is subjected to specific bio-frequencies using a machine, such as the Quantum Alliance Scio EPFX machine. Upon such an exposure, the crystal nature of components found in blood will create a phenomenon known as a “piezoelectric effect” and allow the frequencies used to be captured and retained by components in the blood. The EPFX machine is used to enhance the extracted cells drawn in a syringe and placed on the “charging tray” of the machine with desired charging frequencies added. As a further enhancement, a brief exposure to ultraviolet light (which will not harm healthy cells in the very short period of use, but which will deactivate microorganisms in the mix) is utilized on the stem cells/PRP of each blood draw.

Frequencies have, throughout history, been used to provide bio-enhancement through sound, light, electricity, magnetism, and heat. Each organ of the body works within a specific frequency range for optimal function (i.e. the beta, alpha, delta, gamma frequency range of brain function). To cite an example that has long been accepted in the world of healing, neurologists and neuroscientists have classified the functional states of the brain in terms of frequencies, which are, of course, manifest and measured as Hertz (Hz) or cycles per second. Each brain state, from the very active beta to the more relaxed alpha and the highly relaxed delta and theta, are characterized by measurable frequencies or Hz. This is now a standard of practice. In the same foundational frequency classifications, all organ systems and of course, associated meridians function within a normal frequency range. These have also been measured (i.e.—The optimal frequency, for healthy heart function is between 57-70 Hz, the respiratory and sinus frequencies are between 72-78 Hz, etc.).

In one particular embodiment of the invention, as described above, a frequency imparting machine using electrical frequencies that have been programmed to enhance the cells imparts is used to subject the cells to a particular frequency. With respect to desired optimal function, it has been demonstrated that there is a bio-frequency in each of the 14 meridian systems in the body, as there is a bio-frequency in the “chakras” (considered “the wheels of life”).

Once the fortified, or frequency enhanced, stem cells/PRP have been activated, the first injection in the general systemic circulation is made into a vein in the antecubital fossa or equivalent. The next draw will provide an additional stem cell/PRP mixture. In line with the proven meridian pathways, the stem cell/PRP mixture of the second blood draw will be carefully injected into the desired acupuncture points selected so as to induce travel of the stem cell/PRP mixture to the “target” area or organ (i.e. “liver meridian” considering specific points along it will flow and “open” to the eye (which is its target organ). Therefore, if regeneration is required or desired in relationship to eye and disturbances of the eye, this stem cell/PRP mixture, along with other desired chemicals or biologically desirable and safe components will be placed into acupoints along the “liver meridian” such as the “liver 3 point” or the “liver 2 point”; additionally issues and ailments pertaining to the stomach will require the PRP/stem cell mixture to be injected along the “stomach meridian”, and issues with the kidneys along the “kidney meridian”) utilizing the fact that cellular components along with interstitial fluid will migrate along this meridian system it is expected that the migration will reach the eye within 5-10 minutes and begin to create a healing response in the organ. In one particular embodiment of the invention, a total maximum of 6 points will be used, 2 main points and 4 adjunctive points are the preferred root and, using 1 cc per acupoint, 6 cc will be injected into the meridian.

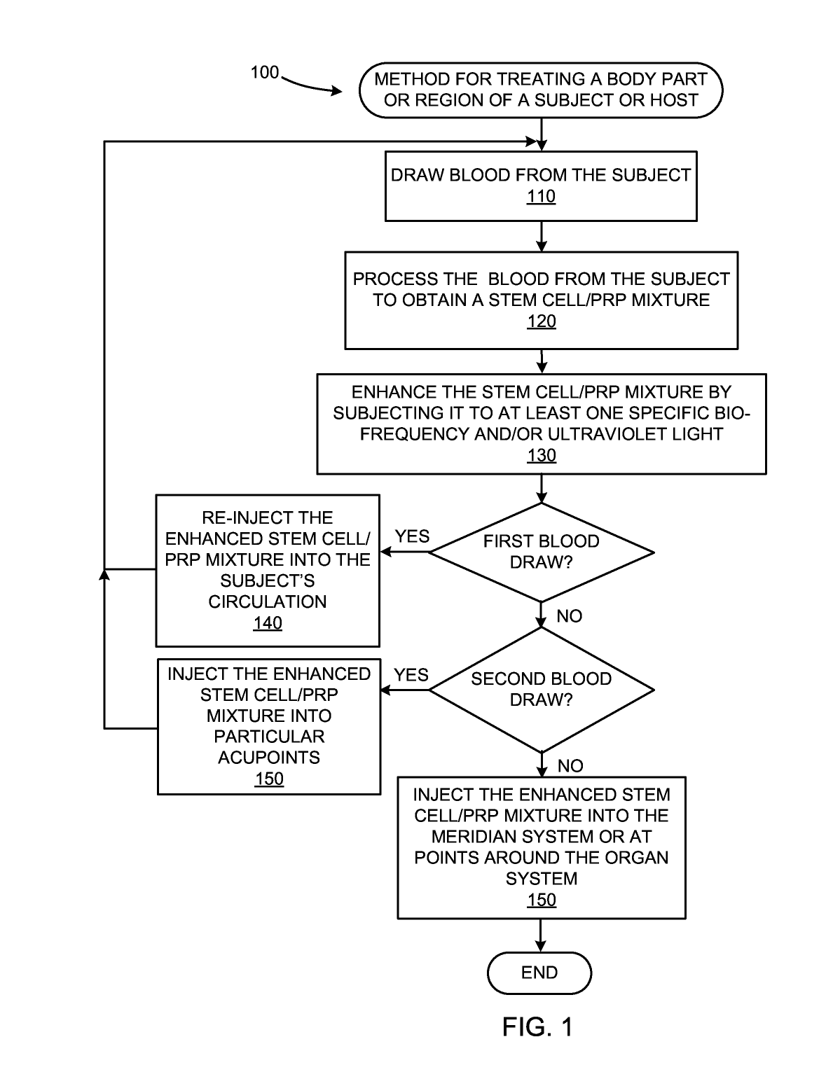

Referring now to FIGS. 1-3, there will be described a method 100 in accordance with one particular embodiment of the invention. First, blood is drawn from the arm vein (or other vein) of the subject or host. Step 110. In the example of the present particular embodiment of the invention, 60 cc of blood will be drawn in each of three separate blood draws. Alternately, as discussed above, stem cells for injection can be obtained from the subject's own fat tissue, using a liposuction technique.

The blood (60 cc) drawn in the first blood draw is placed into an Arteriocyte Megellan Autologous Platelet Separator Machine, or an equivalent machine, in order to produce a desired stem cell/PRP mixture. More particularly, the blood is spun in the separator machine and 8 cc of blood rich in hematopoetic stem cells and PRP are obtained from each 60 cc blood draw. Step 120. If fat tissue is used as the source of autologous cells, the fat tissue is similarly processed i.e., centrifuged and liquefied, to obtain a stem cell mixture.

The 8 cc stem cell/PRP mixture is then further enhanced. Step 130. In accordance with a preferred embodiment, the 8 cc stem cell/PRP mixture is placed on the EPFX Scio Machine to subject it to desired frequencies (in Hertz or Hz) according to an optimal functional frequency of the desired meridian system and corresponding organ being treated. Additionally, in the preferred embodiment, the 8 cc stem cell/PRP mixture is also enhanced by quick exposure to ultraviolet or UV light, to eradicate potential microorganisms in the sample. For purposes of the present application, the term “quick” means an exposure not exceeding 15 seconds. More preferably, the “quick exposure” of the stem cell/PRP mixture to UV light is performed for between 10-15 seconds.

In the present particular embodiment, the first 8 cc of enhanced stem cell/PRP mixture is re-injected into the subject's general circulation (e.g., into a vein of the general circulatory system). Step 140.

Steps 110-130 are then repeated for a second blood draw (or fat tissue harvest), in order to produce a second 8 cc enhanced stem cell/PRP mixture. The second 8 cc enhanced stem cell/PRP mixture is injected into particular desired acupoints found on the meridians associated with the region or organ that is being targeted for therapeutic treatment. Step 150. For example, the second 8 cc enhanced mixture would be injected into acupoints along the liver meridian to treat liver imbalances or in acupoints along the heart meridian to treat heart organ imbalances, etc. In one particular embodiment of the invention, 1-2 cc of the enhanced stem cell/PRP mixture will be injected into each of 4-8 acupoints used. If desired, this second injection can be timed to coincide with the specific two-hour phase when the energy flow is most active in the particular meridian into which the stem cell/PRP mixture is being injected, thus further inducing the cells to go to the targeted organ/region to promote the repair and production of healthy cells in the target organ/region. Additionally, it should be noted that there has been found to be a strong correlation between the locations of classical acupoints and so-called “trigger points” (as coined in 1942 by Dr. Janet Travell). One study has found that 92% of trigger points correspond to acupuncture points [see, for example, Dorsher P T (May 2006). “Trigger points and acupuncture points: anatomic and clinical correlations”. Medical Acupuncture 17 (3)]. As such, for purposes of definition herein, the terms “acupoints” or “acupuncture points” should be interpreted to also mean and include “trigger points” corresponding to known acupoints. For example, it should be understood that, wherein the present application discloses injecting a mixture into a particular acupoint or acupuncture point, it should also be understood that this is the equivalent of, and meant to encompass, an injection of the mixture into a trigger point corresponding to the particular acupoint or acupuncture point.

Steps 110-130 will be again repeated for the third and final blood draw (or fat tissue harvest, if applicable). As with the previously described two blood draws, the final 60 cc blood draw will be spun and enhanced with the appropriate frequencies (i.e. based on fortification of function and elimination of disease) and placed back in either the meridian system or, preferably, in points around the organ system, but which are not part of the meridian system associated with the particular organ or region to be treated. This is called “prolotherapy” in medicine, and it involves the injection of small amounts of substance superficially around the desired area with the belief that the substance will disseminate and flow into the respected region desired. Step 160.

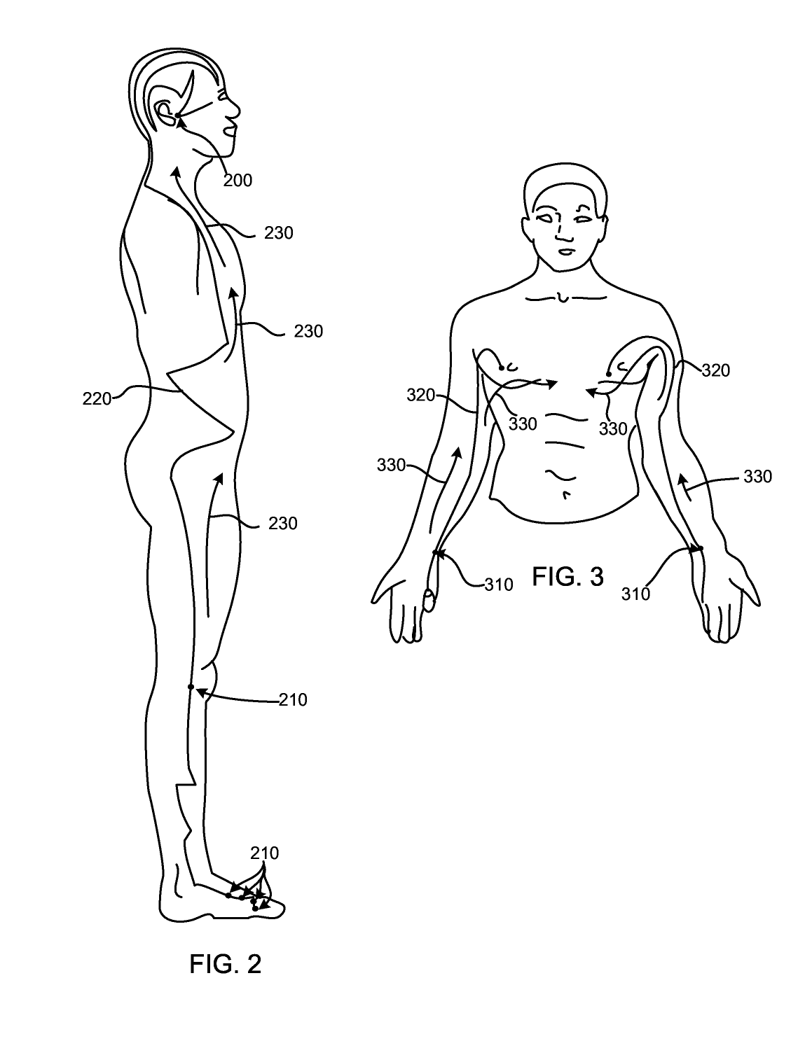

Referring now to FIG. 2, there will be described one particular method of the present invention useful in treating TMJ pain and dysfunction, including headaches caused by TMJ. As discussed in U.S. Patent Application Ser. No. 61/935,118, incorporated herein by reference, TMJ is the abbreviation used for temporomandibular joint, or the joint which connects the jawbone (mandible) to the maxilla of the skull. Aside from the osseous joint structures, the components include a cartilage disc, various tendons, ligaments, nerves and other connective tissues. TMJ is sometimes used as an abbreviation for temporomandibular joint dysfunction syndrome, which is also known as TMD or craniofacial pain dysfunction, as well as other names. Because of the complexity of this joint (it is designed to perform a greater array of functions than all of the other joints in the body), it is readily prone to sustaining tissue injury. TMJ injury can include tissue injury to the supporting and surrounding components of the joint as well.

In the present particular embodiment illustrated in FIG. 2, the use of a stem cell/PRP mixture is used to treat pain and dysfunction of the TMJ 200. More particularly, in the present embodiment, a stem cell/PRP mixture is prepared according to steps 110-120 described above, either from blood drawn from the subject or fat tissue of the subject.

More particularly, one particular embodiment, a blood draw of 60 cc from the antecubital fossa of the patients arm is performed. The drawn blood is mixed with a blood thinner (anticoagulant) and placed in a centrifuge for separation of the stem cell/PRP components from the blood. The blood is placed on the machine, spun and 6-8 cc of stem cell/PRP mixture is obtained. The remainder of the blood is discarded. Preferably, the stem cell/PRP mixture can be enhanced according to step 130, above, as well.

According to the present particular embodiment, the stem cell/PRP mixture is split into five parts and is injected into five acupoints 210 along the gallbladder meridian 220, which is associated with the TMJ 200. In the present particular example illustrated in FIG. 2, five acupoints below the knee on the gall bladder meridian 220—the Yanglingquan, Zulinqi, Siwuhui, Xiaxi and Zuqiaoyin acupoints—were selected to receive injections for treating TMJ. The 6-8 cc mixture is injected into the skin at the selected acupoints 210 after a sterile technique is used to clean the skin.

In one particular embodiment, the subject is injected in each of the designated acupoints 210 with a 1 cc mixture of half and half 2% lidocaine and sarapin. This mixture comprises a 1 cc mix in total. Lidocaine is a local anesthetic which is used to diminish pain in the skin puncture region and sarapin is a plant-derived natural analgesic/anti-inflammatory. Approximately 2-3 minutes after the lidocaine/sarapin injection, each of the designated acupoints 210 will receive 1-2 cc of the stem cell/PRP mixture administered through a 25 gauge needle. Therefore the 6-8 cc mixture should be sufficient for 3-6 regions.

Once the (optionally, enhanced) stem cell/PRP mixture is injected into the selected acupoints 210 in the gall bladder meridian 220, the mixture moves along the gall bladder meridian in the direction of arrows 230, travelling up the body until the mixture reaches the TMJ joint 200 and head region, which is the target area for treating TMJ pain and disfunction.

If desired, the method can be performed with injections only at the acupoints 210. Alternately, the present embodiment can use the method of FIG. 1, wherein a first stem cell/PRP mixture is re-injected into the circulation of the subject, a second stem cell/PRP mixture is injected into the acupoints 210 and a third stem cell/PRP mixture is injected in the face of the patient, as described more particularly in U.S. Patent No. 61/935,118. The foregoing methods can additionally be used to treat headaches using a stem cell/PRP mixture, as described more particularly in connection with U.S. Patent Application Ser. No. 61/944,767, incorporated herein by reference.

Further still, referring now to FIG. 3, there will be described one particular embodiment of a method for treating the heart of a subject using the pericardium meridian. More particularly, a stem cell/PRP mixture is obtained from the subject, in the manner described in connection with FIGS. 1 and 2. Preferably, the stem cell/PRP mixture obtained is also enhanced, as described in connection with step 130 of FIG. 1.

According to the present particular embodiment, the stem cell/PRP mixture is split into two parts and is injected into two acupoints 310 along the pericardium meridian 320, which is associated with the heart. In the present particular example illustrated in FIG. 3, the Daling acupoints on the pericardium meridian 320 were selected to receive injections for treating the heart. The stem cell/PRP mixture is injected into the skin at the selected acupoints 310, as described in connection with FIGS. 1 and 2.

Once the stem cell/PRP mixture is injected into the selected acupoints 310 in the pericardium meridian 320, the mixture moves along the pericardium meridian in the direction of arrows 330, travelling up the body until the mixture reaches the target organ—the heart.

The foregoing treatment can be performed by itself, or in combination with a three part method, as described in connection with FIG. 1, as desired.

Using a combination of Traditional Chinese Medicine, U.V. light, and/or Frequency-enhanced stem cell/PRP mixture combination technique in accordance with the present invention, it is expected that benefits will be provided in the organ being targeted. Such benefits include, but are not limited to, repair, regeneration and rejuvenation, all provided without actually surgically invading the body to physically implant or induce the stem cells to go through the pathway system and reach the target.

Subsequent to treatment by the method described herein, a measurable improvement is expected in the functional integrity in the organ or organs in question, improved physiologic parameters including objective and subjective findings. One benefit of using the method of the invention is that, rather than penetrating an organ such as the heart or the brain (i.e. through the skull surgically or with a needle) and possibly creating potentially significant complications, the present inventive method induces the desired component to travel through the demonstrated meridian system by injecting the mixture just under the skin at designated acupoints. The precise and uniform locations of these acupoints are based on definitive location of joints, folds, creases, etc. and determinable consistently on each individual. The acupoints are areas of lowered skin resistance (which can be measured electrically via commercially available acupoint locating devices). The inventive method is effective for tissue or organ regeneration/rebuilding/repair through the introduction of a host-derived stem cell/PRP mixture (taken from the blood of the host blood and/or mesenchymal organs such as fat or even bone) is introduced through alternative pathways. As described herein, the systemic circulatory pathway is utilized in conjunction to fortify the results.

The present invention described above combines principles of traditional Chinese medicine (utilizing the 24-hour-cycle clock and meridian Qi or interstitial fluid flow coupled with currently available blood stem cell/PRP technologies to introduce cells derived from blood or fat tissue and/or extracted stem cells and PRP into the appropriate acupuncture points, or entrance ramps, into the underground highway of the meridian system. Facilitating the delivery of these cellular products, as well other potential agents, to the desired organ or region initiates rejuvenation, repair and/or growth. The delivery pathway described herein has tremendous potential as a delivery system, not only for stem cells, but potentially other agents as well so that health is restored and human suffering caused by disease or injury is minimized.

The present invention offers multiple benefits in treatment, including the potential for mitigating imbalances in the organ system and/or region being targeted. The technique is based on the utilization of newly confirmed, age-old fluid energy pathways, to bring a frequency enhanced specific component (stem cell/PRP mixture) to a specific desired area in the body without surgically penetrating or penetrating by needle that region. This minimizes potential for post-operative complications while demonstrating great potential as a “targeting” tool to open the door for utilization of other agents, as well. The present invention greatly minimizes the potential for complications and/or reactions, since the patient's own blood components are utilized in the method. Additionally, although described by itself herein, it should be understood that the method of the present invention can additionally be used in conjunction with other therapeutically prescribed modalities currently in use.

Further, the use of frequency enhancement is provided herein in connection with one particularly preferred embodiment of the invention. However, if desired, the method of the present invention can be employed without the added feature of frequency enhancement as well, without departing from the scope and spirit of the present invention. Nevertheless, there is believed to be a strong benefit associated with the use of light enhanced bio-frequency enhancement to the cellular components used in the present invention.

The present invention provides a method combining the use of an enriched cellular mixture (i.e., “a stem cell/PRP mixture) which is placed into the meridian system based on the principles of Traditional Chinese Medicine. Accordingly, while a preferred embodiment of the present invention is shown and described herein, it will be understood that the invention may be embodied otherwise than as herein specifically illustrated or described, and that within the embodiments certain changes in the detail and construction, as well as the arrangement of the parts, may be made without departing from the principles of the present invention as defined by the appended claims.

I claim:

1. A method for treating a particular organ or region of a patient, comprising the steps of:
obtaining a stem cell/platelet-rich-plasma (PRP) mixture from the patient;
identifying a particular meridian associated with the particular organ or region to be treated; and
injecting the enhanced stem cell/PRP mixture into at least one acupoint on the identified meridian.

2. The method of claim 1, further comprising the step of enhancing the stem cell/PRP mixture by subjecting the stem cell/PRP mixture to at least one of a specific frequency or ultraviolet light.

3. The method of claim 2, wherein the stem cell/PRP mixture is subjected to at least one specific bio-frequency associated with the identified particular meridian or with the particular organ or region to be treated.

4. The method of claim 2, wherein the injecting step is performed at a time occurring during a two hour flow cycle associated with the identified particular meridian.

5. The method of claim 1, wherein the stem cell/PRP mixture is obtained from blood of the patient.

6. The method of claim 1, wherein the particular organ or region to be treated is the heart and the identified meridian is the pericardium meridian.

7. The method of claim 1, wherein the particular organ or region to be treated is the temporomandibular joint (TMJ) and the identified meridian is the gall bladder meridian.

8. The method of claim 7, wherein at least five acupoints along the gall bladder meridian are injected with the stem cell/PRP mixture.

9. The method of claim 8, wherein the at least five acupoints selected are all located below the knee of the patient.

10. A method of preparing an enhanced stem cell/PRP mixture for treatment of a particular organ or region of a patient, comprising the steps of:
obtaining a stem cell/PRP mixture from the patient; and
enhancing the obtained stem cell/PRP mixture by subjecting the stem cell/PRP mixture to at least one specific bio-frequency associated with the particular organ or region of the patient.

11. The method of claim 10, further comprising the step of subjecting the stem cell/PRP mixture to ultraviolet (UV) light for no more than 15 seconds.

12. A method of treating a particular organ or region of a patient, comprising the steps of:
providing an enhanced stem cell/PRP mixture according to claim 10; and
injecting the enhanced stem cell/PRP mixture into at least one acupoint along a meridian associated with the particular organ or region of the patient.

13. The method of claim 12, wherein the injecting step is performed at a time occurring during a two hour flow cycle associated with the identified particular meridian.

14. The method of claim 12, wherein the enhanced stem cell/PRP mixture is injected into a plurality of acupoints along the meridian associated with the particular organ or region of the patient.

15. A method of treating a particular organ or region of a patient, comprising the steps of:
obtaining a first stem cell/PRP mixture from the patient;
injecting the first stem cell/PRP mixture into the circulatory system of the patient;
obtaining a second stem cell/PRP mixture from the patient; and
injecting at least a portion of the second stem cell/PRP mixture into at least one acupoint of the patient along a meridian associated with the particular organ or region to be treated.

16. The method of claim 15, further comprising the steps of:
obtaining a third stem cell/PRP mixture from the patient; and
injecting at least a portion of the the third stem cell/PRP mixture into at least one of the meridian system associated with the particular organ or region to be treated or in points around the particular organ or region to be treated which are not along the meridian system associated with the particular organ or region to be treated.

17. The method of claim 16, wherein the third stem cell/PRP mixture is subjected to at least one specific bio-frequency associated with the identified particular meridian or with the particular organ or region to be treated.

18. The method of claim 15, further comprising the step of enhancing the second stem cell/PRP mixture prior to injection by subjecting the stem cell/PRP mixture to at least one of a specific frequency or ultraviolet light.

19. The method of claim 18, wherein the second stem cell/PRP mixture is subjected to at least one specific bio-frequency associated with the identified particular meridian or with the particular organ or region to be treated.

20. The method of claim 15, wherein the second injecting step is performed at a time occurring during a two hour flow cycle associated with the identified particular meridian.
